(19) United States
(12) Patent Application Publication
(10) Pub. No.: US 2007/0078388 A1
(43) Pub. Date: Apr. 5, 2007

(54) Lubricious coating

(75) Inventor: Steve Kangas, Woodbury, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: Boston Scientific Scimed, Inc., Maple Grove, MN (US)

(21) Appl. No.: 11/519,664

(22) Filed: Sep. 12, 2006

Related U.S. Application Data
(63) Continuation of application No. 10/658,718, filed on Sep. 9, 2003, now abandoned.

(51) Int. Cl. A61M 29/00

(52) U.S. Cl. 604/96.01

(57) ABSTRACT

A lubricious coating including at least one ethylenically unsaturated resin and at least one hydrophilic aliphatic polyether polyurethane which does not crosslink with said ethylenically unsaturated resin, and to methods of making and using the same.

### BACKGROUND OF THE INVENTION

The present invention relates to a lubricious composition useful for coatings on medical devices insertable in the body such as catheter assemblies.

Improving the lubricity of insertable medical devices such as by application of lubricious polymeric coatings to the surfaces of such devices for the purpose of reducing friction when the device is introduced into the human body, generally referred to as lubricious coatings, is known in the art.

Catheters and other medical devices used for introduction in blood vessels, urethra, body conduits and the like and guide wires used with such devices are examples of article which may be provided with hydrophilic coatings. Guide catheters, and catheters for balloon angioplasty and biopsy are specific examples of such catheters.

Silicone has been used as a coating for many olefin and metallic medical devices. However, silicone is hydrophobic, and although imparting some lubricity against certain surfaces, silicone's coefficient of friction increases dramatically in the presence of water, plasma, or blood.

Hydrogel polymers have also been used in coatings. Depending on their composition hydrogels are characterized by an initial non-tacky to tacky quality followed by lubricity upon hydration.

### SUMMARY OF THE INVENTION

In one aspect, the present invention relates to a lubricious coating including at least one ethylenically unsaturated and at least one hydrophilic polyurethane.

In another aspect, the present invention relates to a medical device having a lubricious coating, the lubricious coating including at least one ethylenically unsaturated resin and at least one hydrophilic polyurethane.

In one embodiment, the medical device is a catheter device.

The lubricious coating may be used on guide wires, catheter shafts, dilatation balloons, and so forth.

Suitably, the polyurethane is an aliphatic polyether polyurethane.

In some embodiments, the ethylenically unsaturated resin includes at least one mono-, di- or tri-(meth)acrylate.

In one embodiment, a blend of neopentyl glycol diacrylate or polyethylene glycol diacrylate are employed in combination with at least one hydrophilic aliphatic polyether polyurethane. The hydrophilic aliphatic polyether polyurethane may be employed in combination with a second polyurethane polymer which absorbs less water by weight that the hydrophilic aliphatic polyether polyurethane.

The lubricious coatings according to the present invention find utility for reducing frictional forces of insertable medical devices where one surface is movably in contact with another surface.

### BRIEF DESCRIPTION OF THE DRAWINGS

FIG. 1 is a graph showing lubricity and durability of compositions according to the invention as well as comparative examples.

### DETAILED DESCRIPTIONS OF THE PREFERRED EMBODIMENTS

While this invention may be embodied in many different forms, there are described in detail herein specific embodiments of the invention. This description is an exemplification of the principles of the invention and is not intended to limit the invention to the particular embodiments illustrated.

The hydrophilic polyurethanes suitable for use herein are those having a high degree of water absorbancy being capable of absorbing as much as about 500% to about 2000% of their own weight in water.

Suitably, the polyurethane is a thermoplastic polyurethane.

Thermoplastic polyether polyurethanes are a suitable class of polyurethanes, and in particular, aliphatic polyether polyurethanes are suitable for use herein. Examples of such thermoplastic polyurethanes include, but are not limited to, TECOGEL® 500 and TECOGEL® 2000 available from Thermedics, Inc.

Suitable polymers are water swellable, but not water soluble.

Hydrophilic polyurethanes are typically formed with relatively higher amounts of polyethylene oxide or polyethylene glycol.

The highly water absorbent polyurethanes described above, can also be employed in combination with other, less hydrophilic polyurethanes. Examples of suitable polyurethanes are Tecophilic® hydrophilic polyurethanes available from Thermedics, Inc.

Of course, any lubricious polymer may be employed in combination with the hydrophilic polyurethanes described herein. The list of available polymeric materials is vast and such polymeric materials are known to those of ordinary skill in the art.

As used herein, the term ethylenically unsaturated resin, shall be used to refer to any material which has the property of undergoing a chemical reaction which is initiated upon exposure to heat, catalyst, actinic radiation, moisture, etc., to become a relatively insoluble material which, once set, cured or cross-linked, will decompose rather than melt. Typically, such materials referred to herein, may develop a well-bonded three-dimensional structure upon curing.

Any ethylenically unsaturated resin suitable for forming an interpenetrating network (IPN) or semi-interpenetrating network with the hydrophilic polyurethane may be employed herein. Suitably, the crosslinker does not react with the polyurethane.

Suitable radical cure resins include those which are polyfunctional, ethylenically unsaturated compounds such as those under the category of vinyl resins. Examples of suitable resins include, for example, the acrylic esters or acrylates. Examples of such acrylic esters include the (meth)acrylates including mono-, di-, and tri(meth)acrylates and polyacrylates. Examples of suitable members of this class include, but are not limited to, butyl (meth)acrylate, methyl (meth)acrylate, ethyl (meth)acrylate, propyl (meth)acrylate, octyl (meth)acrylate, heptyl (meth)acrylate, nonyl (meth)acrylate, hexyl (meth)acrylate, n-hexyl (meth)acrylate, isopropyl (meth)acrylate, isobutyl (meth)acrylate, decyl (meth)acrylate, isodecyl (meth)acrylate, lauryl (meth)acrylate, stearyl (meth)acrylate, behenyl (meth)acrylate and melissyl (meth)acrylate, methoxyethyl (meth)acrylate, hydroxyl ethyl (meth)acrylate, glycidyl (meth)acrylate, 2-ethylhexyl (meth)acrylate, 2-ethoxyethyl (meth)acrylate, ethylene glycol di(meth)acrylate, propylene glycol di(meth)acrylate, diethylene glycol di(meth)acrylate, polyethylene glycol di(meth)acrylate, 1,5-pentanediol di(meth)acrylate, neopentyl glycol di(meth)acrylate (NPG), 1,6-hexanediol (meth)acrylate, 1,6-hexandiol di(meth)acrylate, polyethylene glycol di(meth)acrylate, polypropylene glycol di(meth)acrylate, pentaerythritol tri(meth)acrylate, trimethylolpropane tri(meth)acrylate, trimethylolpropane dipentaerythritol penta(meth)acrylate, pentaerythritol tetra(meth)acrylate, triethylene glycol di(meth)acrylate, n-butyl (meth)acrylate, benzoin (meth)acrylate, glyceryl propoxy tri(meth)acrylate, 1,3-propylene glycol di(meth)acrylate, tripropylene glycol di(meth)acrylate, 1,3-butylene glycol di(meth)acrylate, 1,4-butanediol di(meth)acrylate, 1,6-hexanediol di(meth)acrylate, diethylene glycol di(meth)acrylate, triethylene glycol di(meth)acrylate, tetraethylene glycol di(meth)acrylate, methyl ethacrylate, ethoxylated bisphenol-A-di(meth)acrylate, and so forth. This list is intended for illustrative purposes only, and is not intended to limit the scope of the present invention. One of ordinary skill in the art would know such materials.

Acrylic nitriles also find utility herein. Examples are the α,β-olefinically unsaturated nitriles including the monoolefinically unsaturated nitriles having from 3 to 10 carbon atoms such as acrylonitrile, methacrylonitrile, and the like.

Illustrative amides include acrylamide, methacrylamide, N-t-butyl acrylamide, N-cyclohexyl acrylamide, methylene-bis-acrylamide, trimethylene-bis-acrylamide, hexamethylene-bis-acrylamide, N,N-dimethylacrylamide and N,N-diethylacrylamide, m-phenylene-bis-acrylamide, p-phenylene-bis-acrylamide, N-methylol-acrylamide, diacetone-acrylamide, butoxymethyl acrylamide, and so forth.

N-alkylol amides of α,β-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like find utility herein.

(Meth)acrylic acids find utility herein.

Other examples include, but are not limited to, N-acrylamido-morpholine, N-acrylamido-piperidine, acrylic acid anilide, methacrylic acid anilide, divinyl benzene, styrene, methyl styrene, butadiene, isoprene, vinyl functional silicones, chlorostyrene, methoxystyrene, chloromethylstyrene, vinyl toluene, 1-vinyl-2-methylimidazole, 1-vinyl-2-undecylimidazole, 1-vinyl-2-undecylimidazoline, N-vinylpyrrolidone, N-vinylcarbazole, vinylbenzyl ether, bis(4-acryloxypolyethoxyphenyl)propane, vinyl ethers, vinylphenyl ether, vinyl esters, carboxylic acids, N,N′-diacrylamidopiperazine, pentaerythritol tetra-allyl ether, and so forth, to mention only a few.

Suitable resins are described in EP 0 363 460 B1, U.S. Pat. No. 4,051,195, U.S. Pat. No. 2,895,950, U.S. Pat. No. 3,218,305, U.S. Pat. No. 3,425,988, U.S. Pat. No. 5,693,034, U.S. Pat. No. 6,558,798, U.S. Pat. No. 6,583,214, for example, each of which is incorporated by reference herein in its entirety.

Any suitable copolymers of the above-described compounds with other monomers containing polymerizable vinyl groups also find utility herein.

The amount and types of resins that may be employed are too vast to list. Thus, the above lists are intended for illustrative purposes only, and are not intended to limit the scope of the present invention. Other suitable materials would also find utility herein. Such materials are known to those of ordinary skill in the art.

Other examples include, but are not limited to, thermoset resins such as epoxies, unsaturated polyesters, and isocyante based prepolymers.

The above-described ethylenically unsaturated resins may include both one-part and two-part systems, although the one-part systems are desirably employed herein.

In preparing the solution mixture of the present invention, the hydrophilic polyurethane may be mixed with the ethylenically unsaturated resin in a solvent or cosolvent mixture. Examples of suitable organic solvents of a more polar nature include, but are not limited to, the lower alcohols including, but not limited to, isopropyl alcohol and methanol; water; linear or cyclic carboxamides such ad N,N-dimethylacetamide (DMAC), N,N-diethylacetamide, dimethylformamnide (DMF), diethylformamide or 1-methyl-2-pyrrolidone (NMP); dimethylsulphoxide (DMSO); and so forth.

Other suitable organic solvents include, but are not limited to, aliphatic, cycloaliphatic or aromatic ether-oxides, more particularly dipropyl oxide, diisopropyl oxide, dibutyl oxide, methyltertiobutylether, ethylene glycol dimethylether (glyme), diethylene glycol dimethylether (diglyme); phenyl oxide; dioxane, tetrahydrofuran (THF). Of course, mixtures of solvents may also be employed.

The above lists are intended for illustrative purposes only and are not intended to limit the scope of the present invention. Other solvents not listed herein would find utility in the invention as well and are known to those of skill in the art.

Crosslinking for UV curable compositions may be facilitated by the addition of a small amount of a photoinitiator such as a free radical initiator or cationic photoinitiators as are commonly used for UV curing. Examples of suitable photoinitiators include, but are not limited to, aromatic-aliphatic ketone derivatives, including benzoin and its derivatives, 2-phenyl-1-indanone, and so forth.

Specific examples of a useful photoinitiator include, but are not limited to, 2,2′ dimethoxy-2-phenylacetophenone (IRGACURE® 651), 1-benzoyl-2-hydroxy propane (DAROCUR® 1173), a morpholinoketone (IRGACURE® 369), a bisacylphosphine oxide (IRGACURE® 819), all available from Ciba® Specialty Chemicals, and 2,4,6 dimethylbenzoyl(diphenyl)phosphine oxide (LUCIRIN® TPO available from BASF).

The mixture may then be applied to a substrate out of solvent. The lubricious coating may then be coated onto a surface out of solvent using any coating method known in the art such as dipping, spraying, painting, sponge coating, and so forth.

Crosslinkers which have a higher molecular weight and which are not highly volatile, can be compounded directly with a thermoplastic polyurethane, allowing for coextrusion of the coating.

The solvent may then be allowed to dry. The coating may be dried at room temperature. However, improved durability may be achieved by drying the coating at elevated temperatures of, for example, 70° C. Suitably, drying is conducted at an elevated temperature over several hours to improve the durability of the coating. Once a coating has been applied to a substrate, the coating may then be crosslinked by exposing the coating to heat or actinic radiation such as UV light for a short period of time. This can then trigger the polymerization and crosslinking of the ethylenically unsaturated resin or prepolymer. Suitably the mixture is cured using a high intensity ultraviolet lamp.

The crosslinked structure helps to retain the hydrophilic polyurethane on surfaces to which the coating is applied.

The lubricious coatings according to the invention find utility in the medical device industry, in particular for medical devices inserted in the body. For example, the lubricious coatings find utility on catheter devices, in particular, on guide wires, catheter shafts, dilatation balloons, and so forth.

Dilatation balloons may be coated on the body, cone and/or waist portions or any combination thereof. In some embodiments, the balloon is coated on the distal and proximal waist cones, and on a portion of the body, but not in the center of the body. This has been found to reduce “watermelon seeding”, a term of art used to refer to slippage of the balloon during inflation in a lesion. This can be an issue in particular when the lesion is tapered, but this is not the only situation where “watermelon seeding” can occur.

The lubricity of the coating may be controlled by adding different polyurethanes or other polymers to the blend. This can allow for the use of different coatings on different portions of a catheter device where higher or lower lubricity may be desirable. For example, it may be desirable to coat the proximal portion of the catheter device with a less lubricious formula for better gripping, and to coat the distal portion of the device with a more highly lubricious coating for better trackability. This may be advantageous for guide wires or PV catheter assemblies.

In one embodiment, the distal portion is coated with a ethylenically unsaturated resin and a highly water absorbent aliphatic polyether polyurethane and the proximal portion is coated with a ethylenically unsaturated resin and a blend of a highly water absorbent thermoplastic aliphatic polyether polyurethane and a less water absorbent polymer such as a less water absorbent polyurethane.

The coating according to the present specification may be employed for drug delivery. A drug can be incorporated into the polymer network formed by the crosslinked material which helps to entrap a drug(s) which can then more slowly leach out of the crosslinked network when the medical device is employed in the body.

The following non-limiting examples further illustrate the present invention.

### EXAMPLES

### Example 1

TECOGEL® 2000 polyether polyurethane available from Thermedics, Inc. and neopentylglycol diacrylate (NPGDA (700 MW)) (90/10) was added to a cosolvent blend of isopropyl alcohol (IPA) and water to prepare a 5% solution of TECOGEL® 2000 and NPGDA in 3.75 IPA:1 water. IRGACURE® 369 photoinitiator was added at a 2% loading.

### Example 2

TECOGEL® 2000 polyether polyurethane and polyethyleneglycol diacrylate (PEGDA) (90/10) was added to a cosolvent blend of isopropyl alcohol (IPA) and water to prepare a 5% solids mixture of TECOGEL® 2000 and PEGDA in 3.75 IPA:1 water. IRGACURE® 369 photoinitiator was added at a 2% loading.

### Comparative Example A

A mixture of, polyethylene oxide having a molecular weight of about 90,000 g/mole and NPGDA (10:1) in a cosolvent blend of 3.75:1 isopropyl alcohol (IPA) to water was used to form a 2% solids mixture in solvent. The mixture was applied to a balloon formed of PEBAX® 7033 as described above. Azobisisibutironitrile photoinitiator (2%) was also added in a minimal amount effective to initiate NPG polymerization. This composition is a standard in the industry.

A 2% solids mixture was employed for comparative A versus examples 1 and 2 due to the fact that a 5% solids mixture of examples 1 and 2 is comparable in coating thickness to a 2% solids mixture of comparative A. The molecular weight of TECOGEL® 2000 requires a higher solids content to attain the same coating thickness because it has a lower viscosity than the polyethylene oxide employed in comparative example A.

Each of the above coating compositions were sponge coated on helium plasma treated catheter shafts formed from Pebax 7233 and allowed to dry for several minutes at room temperature. The coatings were cured for 30 sec using a Hg vapor arc lamp.

### Comparative Example B

A 5% solids solution of TECOGEL® 2000 was prepare in a cosolvent blend of 3.75:1 IPA to water. No crosslinker was employed. This solution was applied to a dilatation balloon formed form PEBAX® 7033 polyether block amide copolymer. The coating was allowed to dry at room temperature for 1 hour and 45 minutes.

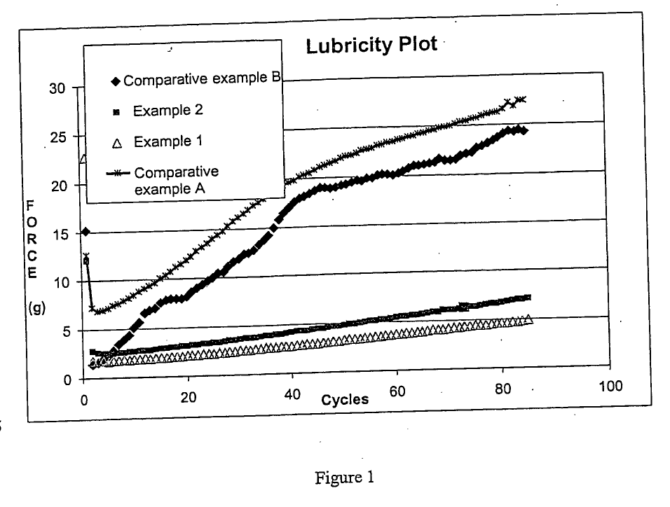

Lubricity was measured using a device that cycles a latex pad along the length of a catheter. The catheter was immersed in water. The latex pad was affixed to an armature which was further connected to a force gauge. An 80 g weight was placed on the armature. The catheter was then cycled back and forth across the pad by a motor drive. Force was measured as a function of the number of cycles. The lower the force, the greater the lubricity. The results are shown in FIG. 1.

The lubricity of comparative examples A and B was initially good, but exhibited poor durability.

Addition of NPGDA or PEGDA to the TECOGEL® 2000 polyurethane showed significant improved in both initial lubricity as well as in durability, i.e. final lubricity which was 5-6 grams. This is due to enhancement to the durability of the polyurethane by entanglement of the themoplastic polyurethane with the cross-linked acrylate network (semi-IPN).

The above disclosure is intended to be illustrative and not exhaustive. The description will suggest many variations and alternatives to those of ordinary skill in the art. All of these alternatives and variations are intended to be included within the scope of the attached claims. Those familiar with the art may recognize other equivalents to the specific embodiments described herein which equivalents are also intended to be encompassed by the claims attached hereto.

1-41. (canceled)

42. A catheter assembly comprising at least one catheter shaft having a proximal end and a distal end, the catheter shaft formed from a first polymer composition, and at least one expandable balloon member disposed about the distal end of the catheter shaft, the expandable medical balloon member formed from a second polymer composition, at least a portion of the catheter assembly comprising a lubricious coating, the lubricious coating formed from a third polymer composition different than the first and second polymer compositions, the third polymer composition is an interpenetrating or semi-interpenetrating polymer network comprising at least one ethylenically unsaturated resin and at least one hydrophilic thermoplastic aliphatic polyether polyurethane wherein the lubricious coating is cured.

43. The catheter assembly of claim 42 wherein said thermoplastic aliphatic polyether polyurethane is substantially uncrosslinked.

44. The catheter assembly of claim 42 wherein said expandable medical balloon comprises said lubricious coating.

45. The catheter assembly of claim 44 wherein said expandable medical balloon is formed from poly(ether-block-amide).

46. The catheter assembly of claim 42 wherein said hydrophilic thermoplastic aliphatic polyether polyurethane absorbs about 500% to about 2000% of its own weight in water upon exposure to an aqueous environment.

47. The catheter assembly of claim 42 wherein said at least one ethylenically unsaturated resin comprises functional groups which are activated photochemically.

48. The catheter assembly of claim 42 further comprising at least one photoinitiator.

49. The catheter assembly of claim 42 wherein said at least one ethylenically unsaturated resin comprises functional groups which are activatable by ultraviolet radiation.

50. The catheter assembly of claim 42 wherein said at least one ethylenically unsaturated resin comprises at least one member selected from the group consisting of mono-, di- and tri-acrylates, polyacrylates and mixtures thereof.

51. The catheter assembly of claim 42 wherein said at least one ethylenically unsaturated resin is a diacrylate.

52. The catheter assembly of claim 50 wherein said at least one ethylenically unsaturated resin comprises at least one member selected from the group consisting of butyl (meth)acrylate, methyl (meth)acrylate, ethyl (meth)acrylate, propyl (meth)acrylate, octyl (meth)acrylate, heptyl (meth)acrylate, nonyl (meth)acrylate, hexyl (meth)acrylate, n-hexyl (meth)acrylate, isopropyl (meth)acrylate, isobutyl (meth)acrylate, decyl (meth)acrylate, isodecyl (meth)acrylate, lauryl (meth)acrylate, stearyl (meth)acrylate, behenyl (meth)acrylate and melissyl (meth)acrylate, methoxyethyl (meth)acrylate, hydroxyl ethyl (meth)acrylate, glycidyl (meth)acrylate, 2-ethylhexyl (meth)acrylate, 2-ethoxyethyl (meth)acrylate, ethylene glycol di(meth)acrylate, propylene glycol di(meth)acrylate, diethylene glycol di(meth)acrylate, polyethylene glycol di(meth)acrylate, 1,5-pentanediol di(meth)acrylate, neopentyl glycol di(meth)acrylate (NPG), 1,6-hexanediol (meth)acrylate, 1,6-hexandiol di(meth)acrylate, polyethylene glycol di(meth)acrylate, polypropylene glycol di(meth)acrylate, pentaerythritol tri(meth)acrylate, trimethylolpropane tri(meth)acrylate, trimethylolpropane dipentaerythritol penta(meth)acrylate, pentaerythritol tetra(meth)acrylate, triethylene glycol di(meth)acrylate, n-butyl (meth)acrylate, benzoin (meth)acrylate, glyceryl propoxy tri(meth)acrylate, 1,3-propylene glycol di(meth)acrylate, tripropylene glycol di(meth)acrylate, 1,3-butylene glycol di(meth)acrylate, 1,4-butanediol di(meth)acrylate, 1,6-hexanediol di(meth)acrylate, diethylene glycol di(meth)acrylate, triethylene glycol di(meth)acrylate, tetraethylene glycol di(meth)acrylate, methyl ethacrylate, ethoxylated bisphenol-A-di(meth)acrylate and mixtures thereof.

53. The catheter assembly of claim 42 wherein said at least one ethylenically unsaturated resin is selected from the group consisting of neopentyl glycol diacrylate, polyethylene glycol diacrylate and mixtures thereof.

54. A guide wire comprising a lubricious coating, the lubricious coating is an interpenetrating or semi-interpenetrating polymer network comprising at least one ethylenically unsaturated resin and at least one hydrophilic thermoplastic aliphatic polyether polyurethane wherein the lubricious coating is cured.

55. The guide wire of claim 54 wherein said hydrophilic thermoplastic aliphatic polyether polyurethane absorbs about 500% to about 2000% of its own weight in water upon exposure to an aqueous environment.

56. The guide wire of claim 54 wherein said at least one ethylenically unsaturated resin comprises functional groups which are activated photochemically.

57. The guide wire of claim 54 further comprising at least one photoinitiator.

58. The guide wire of claim 54 wherein said at least one ethylenically unsaturated resin comprises functional groups which are activatable by ultraviolet radiation.

59. The guide wire of claim 54 wherein said at least one ethylenically unsaturated resin comprises at least one member selected from the group consisting of mono-, di- and tri-acrylates, polyacrylates and mixtures thereof.

60. The guide wire of claim 54 wherein said at least one ethylenically unsaturated resin is a diacrylate.

61. The guide wire of claim 59 wherein said at least one ethylenically unsaturated resin comprises at least one member selected from the group consisting of butyl (meth)acrylate, methyl (meth)acrylate, ethyl (meth)acrylate, propyl (meth)acrylate, octyl (meth)acrylate, heptyl (meth)acrylate, nonyl (meth)acrylate, hexyl (meth)acrylate, n-hexyl (meth)acrylate, isopropyl (meth)acrylate, isobutyl (meth)acrylate, decyl (meth)acrylate, isodecyl (meth)acrylate, lauryl (meth)acrylate, stearyl (meth)acrylate, behenyl (meth)acrylate and melissyl (meth)acrylate, methoxyethyl (meth)acrylate, hydroxyl ethyl (meth)acrylate, glycidyl (meth)acrylate, 2-ethylhexyl (meth)acrylate, 2-ethoxyethyl (meth)acrylate, ethylene glycol di(meth)acrylate, propylene glycol di(meth)acrylate, diethylene glycol di(meth)acrylate, polyethylene glycol di(meth)acrylate, 1,5-pentanediol di(meth)acrylate, neopentyl glycol di(meth)acrylate (NPG), 1,6-hexanediol (meth)acrylate, 1,6-hexandiol di(meth)acrylate, polyethylene glycol di(meth)acrylate, polypropylene glycol di(meth)acrylate, pentaerythritol tri(meth)acrylate, trimethylolpropane tri(meth)acrylate, trimethylolpropane dipentaerythritol penta(meth)acrylate, pentaerythritol tetra(meth)acrylate, triethylene glycol di(meth)acrylate, n-butyl (meth)acrylate, benzoin (meth)acrylate, glyceryl propoxy tri(meth)acrylate, 1,3-propylene glycol di(meth)acrylate, tripropylene glycol di(meth)acrylate, 1,3-butylene glycol di(meth)acrylate, 1,4-butanediol di(meth)acrylate, 1,6-hexanediol di(meth)acrylate, diethylene glycol di(meth)acrylate, triethylene glycol di(meth)acrylate, tetraethylene glycol di(meth)acrylate, methyl ethacrylate, ethoxylated bisphenol-A-di(meth)acrylate and mixtures thereof.

62. The guide wire of claim 54 wherein said at least one ethylenically unsaturated resin is selected from the group consisting of neopentyl glycol diacrylate, polyethylene glycol diacrylate and mixtures thereof.
